(19) United States
(12) Patent Application Publication
(10) Pub. No.: US 2012/0001098 A1
(43) Pub. Date: Jan. 5, 2012

(54) EXCIMER RADIATION LAMP ASSEMBLY, AND SOURCE MODULE AND FLUID TREATMENT SYSTEM CONTAINING SAME

(75) Inventors: Jim Fraser, St. Thomas (CA); Michael Sasges, Victoria (CA)

(73) Assignee: TROJAN Technologies Inc., London (CA)

(21) Appl. No.: 13/176,633

(22) Filed: Jul. 5, 2011

Related U.S. Application Data
(63) Continuation of application No. 12/158,817, filed on Dec. 23, 2008, now abandoned.
(60) Provisional application No. 60/752,024, filed on Dec. 21, 2005.

(51) Int. Cl. H01J 61/30

(52) U.S. Cl. 250/504 R

(57) ABSTRACT

An excimer radiation lamp assembly. The lamp assembly comprises a radiation emitting region and at least one substantially radiation opaque region. The radiation emitting region comprises a pair of dielectric elements disposed in a substantially coaxial arrangement.

### CROSS REFERENCE TO RELATED APPLICATIONS

This application is a continuation of U.S. patent application Ser. No. 12/158,817, filed Dec. 23, 2008, which is a 371 of International Application No. PCT/CA2006/002154, filed Dec. 21, 2006. International Application No. PCT/CA2006/002154, filed Dec. 21, 2006, also claims benefit of U.S. patent application Ser. No. 60/752,024, filed Dec. 21, 2005. The contents of all of these documents are incorporated herein by reference.

### FIELD OF THE INVENTION

In one of its aspects, the present invention relates to an excimer radiation lamp assembly. In another of its aspects, the present invention relates to a radiation source module comprising the excimer radiation lamp assembly. In another of its aspects, the present invention relates to a fluid treatment system comprising the excimer radiation lamp assembly.

### DESCRIPTION OF THE RELATED ART

Fluid treatment systems are known generally in the art.

For example, U.S. Pat. Nos. 4,482,809, 4,872,980, 5,006,244, 5,418,370, 5,539,210 and Re:36,896 (all in the name of Maarschalkerweerd and all assigned to the assignee of the present invention) all describe gravity fed fluid treatment systems which employ ultraviolet (UV) radiation.

Generally, such prior fluid treatment systems employ an ultraviolet radiation lamp to emit radiation of a particular wavelength or range of wavelengths (usually between 185 and 400 nm) to effect bacterial kill or other treatment of the fluid being treated. Many conventional ultraviolet radiation lamps are known as “low pressure” mercury lamps.

In recent years, the art in low pressure mercury lamps has evolved with the development of the so-called Low Pressure, High Output (LPHO) and amalgam UV radiation lamps. These lamps have found widespread use in UV radiation water treatment systems, particularly those used for treatment of municipal drinking water and wastewater. As used herein, the term “low pressure” UV radiation lamp is intended to encompass conventional UV radiation lamps, LPHO UV radiation lamps and amalgam UV radiation lamps.

Low pressure UV radiation lamps and medium pressure UV radiation lamps are the current standard used for UV radiation treatment of municipal drinking water and wastewater.

In recent years, there has been development in the area of so-called excimer radiation lamps. These lamps have the potential to be used in a variety of applications. One such application is UV radiation treatment of water—e.g., municipal drinking water and wastewater.

To date, there has been little or no development of excimer radiation lamps for use in the UV radiation treatment of water—e.g., municipal drinking water and wastewater.

Accordingly, there is a real need in the art for an excimer radiation lamp that is well suited for use in the UV radiation treatment of water—e.g., municipal drinking water and wastewater. In a similar vein, there is a need in the art for a radiation source module and a fluid treatment system incorporating such an excimer radiation lamp.

### SUMMARY OF THE INVENTION

It is an object of the present invention to provide a novel radiation excimer radiation lamp assembly.

It is a further object of the invention to provide a novel radiation source module.

It is yet a further object of the present invention to provide a novel fluid treatment system.

Accordingly, in one of its aspects, the present invention provides an excimer radiation lamp assembly comprising a radiation emitting region and at least one substantially radiation opaque region, the radiation emitting region comprising a pair of dielectric elements disposed in a substantially coaxially arrangement.

In another of its aspects, the present invention provides an excimer radiation lamp assembly comprising a radiation emitting region and an electrode in electrical connection with the radiation emitting region, at least a portion of the radiation emitting region comprising a substantially radiation opaque element independent of the electrode.

In yet another of its aspects, the present invention provides an excimer radiation lamp assembly comprising an elongate cylindrical radiation emitting region and a substantially radiation opaque region, the elongate cylindrical radiation emitting region and the substantially radiation opaque region comprising substantially the same outer diameter.

In yet another of its aspects, the present invention provides a liquid immersible elongate excimer radiation lamp assembly having a longitudinal dimension, the assembly comprising:

a first end and a second end opposed to the first end;

a first region interposed between the first end and the second end for emission of a radiation having a prescribed wavelength; and

a second region juxtaposed with respect to the first region, the second region being radiation opaque or for emission of radiation different than the prescribed wavelength;

wherein the first end has at least one cross-sectional dimension different than the second end.

In yet another of its aspects, the present invention relates to an excimer radiation lamp assembly comprising a radiation emitting region and at least one substantially radiation opaque region, the radiation emitting region comprising a dielectric element and an electrode disposed in a substantially coaxial arrangement.

In yet another of its aspects, the present invention relates to a radiation source module comprising the present excimer radiation lamp assembly.

In yet another of its aspects, the present invention relates to a fluid treatment system comprising the present excimer radiation lamp assembly.

In a highly preferred embodiment the present excimer radiation lamp assembly is configured so as to emit ultraviolet radiation.

### BRIEF DESCRIPTION OF THE DRAWINGS

Embodiments of the present invention will be described with reference to the accompanying drawings, wherein like reference numerals denote like parts, and in which:

FIGS. 1-7 illustrate various views of a first embodiment of the present excimer radiation lamp assembly.

FIGS. 8-12 and 16-18 illustrate various views of a second preferred embodiment of the present excimer radiation lamp assembly.

FIGS. 13-15 illustrate various views of a third preferred embodiment of the present excimer radiation lamp assembly.

FIGS. 19-23 illustrate a fourth embodiment of the present excimer radiation lamp assembly.

FIGS. 24-27 illustrate a fifth embodiment of the present excimer radiation lamp assembly.

FIGS. 28-30 illustrate a sixth embodiment of the present excimer radiation lamp assembly.

FIGS. 31-36 illustrate implementation of embodiments of the present excimer radiation lamp assembly in a radiation source module.

### DETAILED DESCRIPTION OF THE PREFERRED EMBODIMENTS

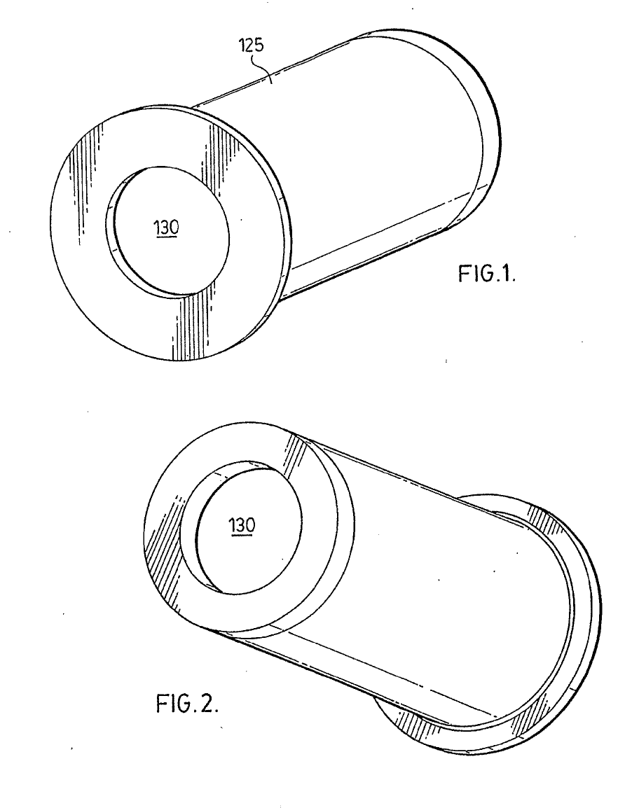

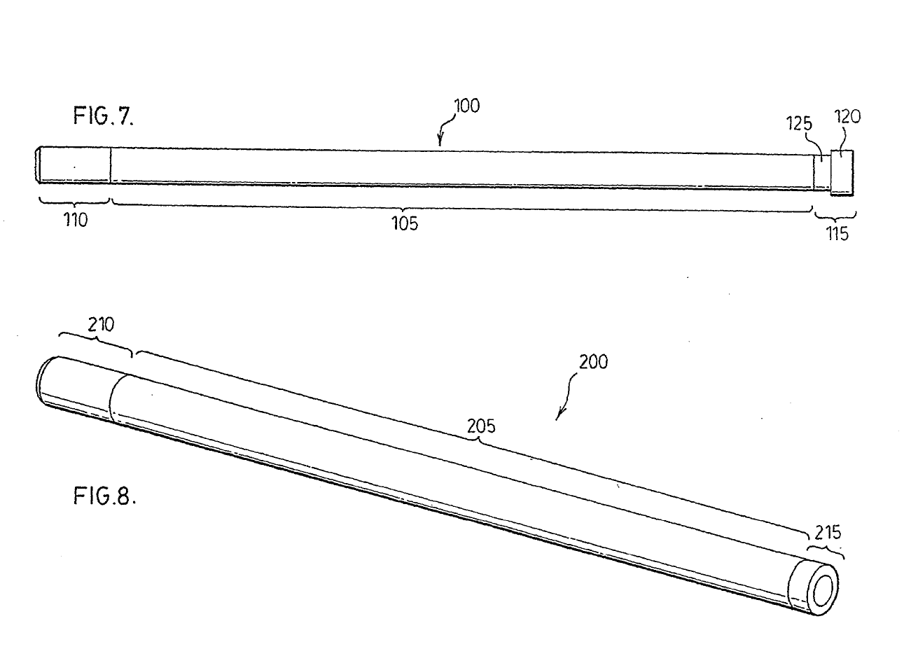

With reference to FIGS. 1-7, there is illustrated an excimer radiation lamp assembly 100 comprising a radiation emitting region 105, a first radiation opaque region 110 and second radiation opaque region 115.

First radiation opaque region 110 may be suitably sized to serve as a parking spot for a cleaning system (not shown) used to remove undesirable materials from the surface of radiation emitting region 105.

Radiation opacity may be conferred to region 110 by any suitable means. For example, it is possible to apply a coating to the appropriate region of lamp assembly 100 which serves to confer radiation opacity to that region.

Alternatively, it is possible to use a radiation opaque element secured to the appropriate region of lamp assembly 100. Non-limiting examples of such radiation opaque elements may be selected from the group consisting of ceramic, rubber, plastic, wood and mixtures thereof.

The provision of region 110 provides a suitable parking location for a cleaning system whereby the seals and other components of the cleaning system will be less likely to damage and/or failure from exposure to radiation.

Radiation opaque region 115 comprises an end portion 120 having a relatively large diameter and a radiation opaque element 125. Preferred embodiments of region 115 are shown in FIGS. 1-3.

FIG. 1 illustrates an enlarged perspective view of end region 115 comprises an opening 130 receiving an electrode (not shown) conventionally used in excimer radiation lamp assemblies.

FIG. 2 is a view of FIG. 1 at the opposite end thereof.

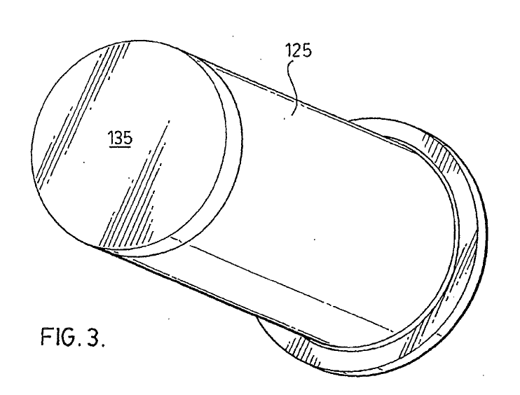

FIG. 3 is a modification of the embodiment shown in FIG. 1 whereby a dome or a cover element 135 is placed between radiation emitting region 105 and end portion 120.

The important point is that end region 115 contains a radiation opaque region which serves to protect the seals and other components of the radiation lamp assembly and/or its surrounding environment.

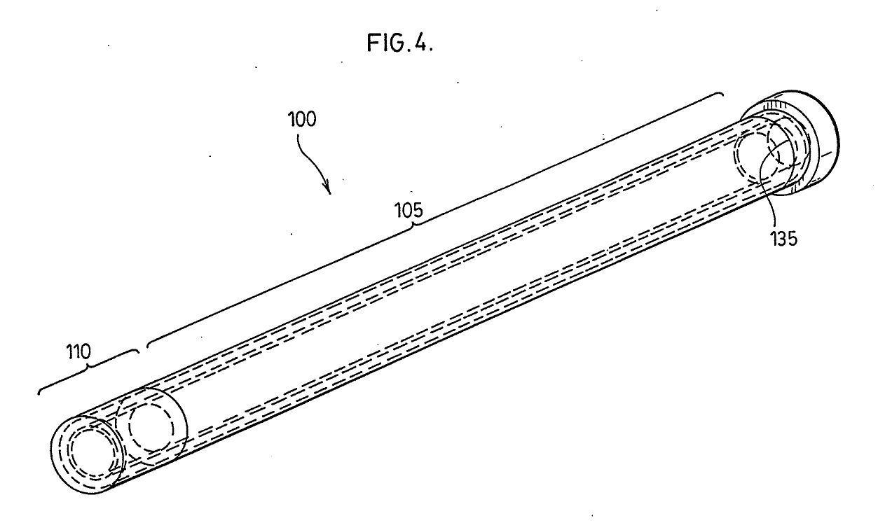

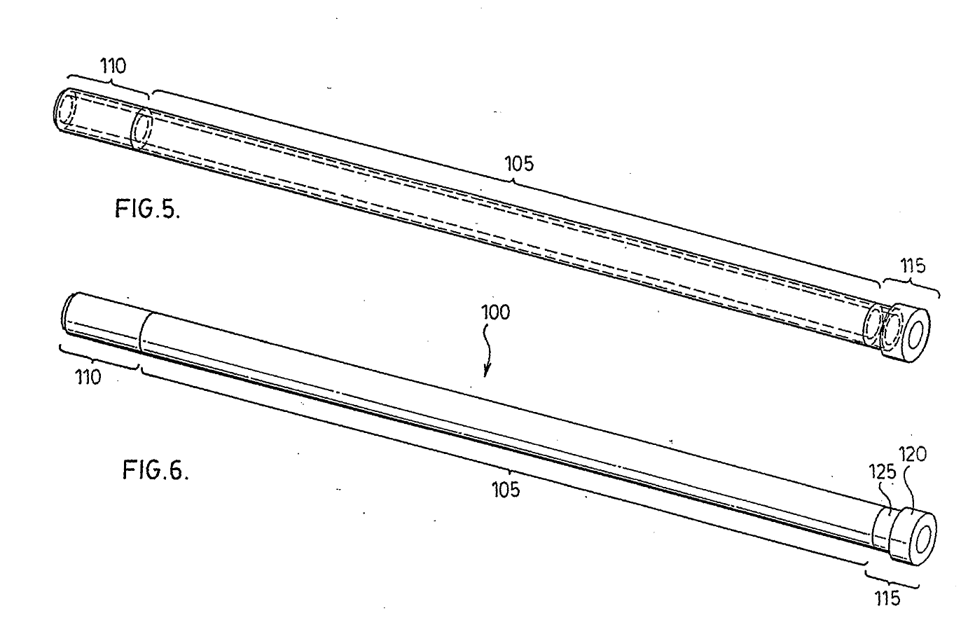

FIG. 4 illustrates implementation of the embodiment illustrated in FIG. 3 whereas FIG. 5 illustrates implementation of the embodiments illustrated in FIGS. 1 and 2. FIGS. 4 and 5 show a ghosted outline of the internal design of an otherwise conventional excimer radiation lamp assembly.

The provision of regions 110 and 115 serve to protect components and other accessories used with the lamp in a fluid treatment system from damage owing to radiation exposure. Further, by providing a larger diameter structure in region 115, radiation lamp assembly 100 is effectively “keyed” so that it can be installed in a unidirectional manner.

With reference to FIGS. 8-12 and 16-18, there is shown an excimer radiation lamp assembly 200.

In the subsequent figures of the present application, the last two digits in a reference numeral are intended to denote a similar element as that shown in the embodiment for FIGS. 1-7. Thus, radiation opaque region 115 in FIGS. 1-7 is similar to radiation opaque element 215 in the embodiment shown in FIGS. 8-12 and 16-18, etc.

The embodiment shown in FIGS. 8-12 and 16-18 is similar to that shown in FIGS. 1-7 with the exception that a larger diameter element is not provided in region 215 of excimer radiation lamp assembly 200.

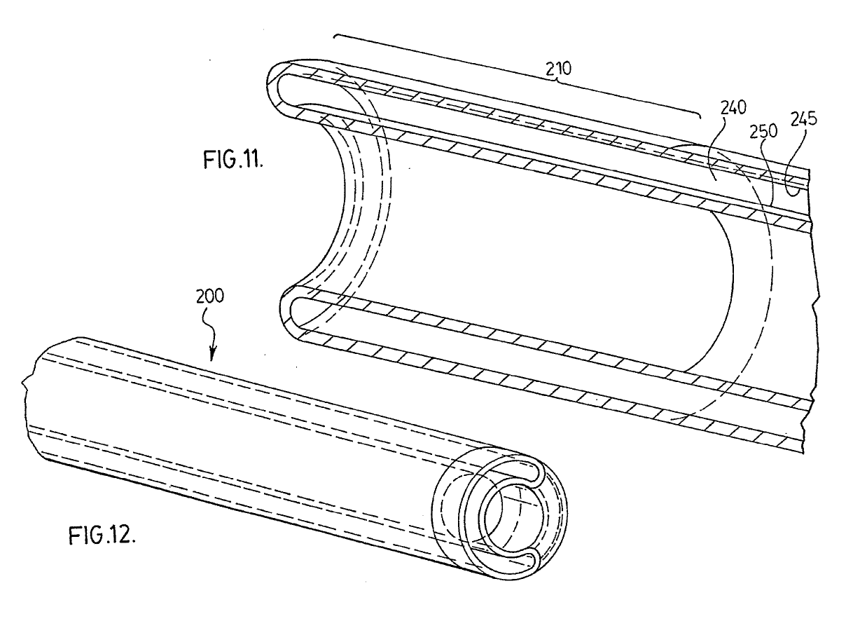

With reference to FIGS. 10-12, additional detail is given on the design of radiation lamp assembly 200. Thus, as is conventional in art of excimer radiation lamps, an annular chamber 240 is provided. A phosphor material (not shown) may be applied to one or both, preferably both of surfaces 245 and 250 of annular chamber 240.

With regard to radiation opaque region 210, radiation opacity may be conferred to this region as discussed above by applying suitable radiation opaque material to the outer and/or inner surfaces of annular chamber 240 corresponding to radiation opaque region 210.

The embodiment shown in FIG. 12 extends annular chamber 240 partially to the end of radiation lamp assembly 200.

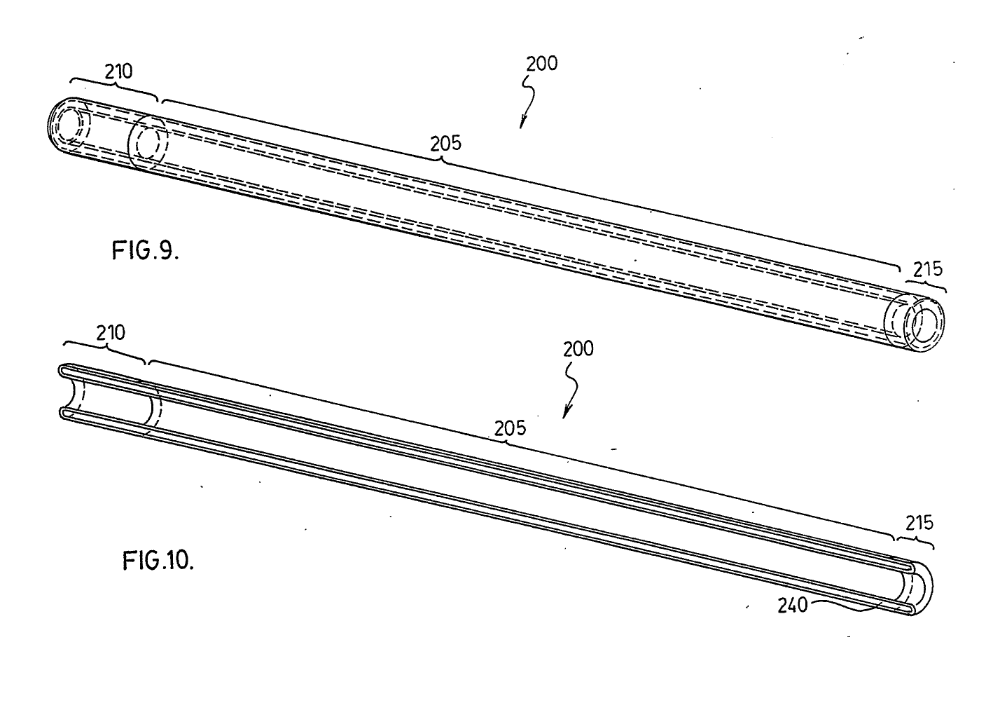

With reference to FIGS. 16 and 17, these Figures show a side elevation with ghosted lines of the embodiment illustrated in FIGS. 8 and 9.

FIG. 17 illustrates a cross-section of the embodiment shown in FIG. 16.

The embodiment shown in FIG. 18 is a slight modification of that shown in the earlier figures. Specifically, in the embodiment shown in FIG. 18, radiation opaque region 210 is of the same size as radiation opaque region 215. This embodiment is particularly well suited to the situation where a cleaning system (not shown) can suitably clean the exterior of radiation emitting region 205 in a single stroke.

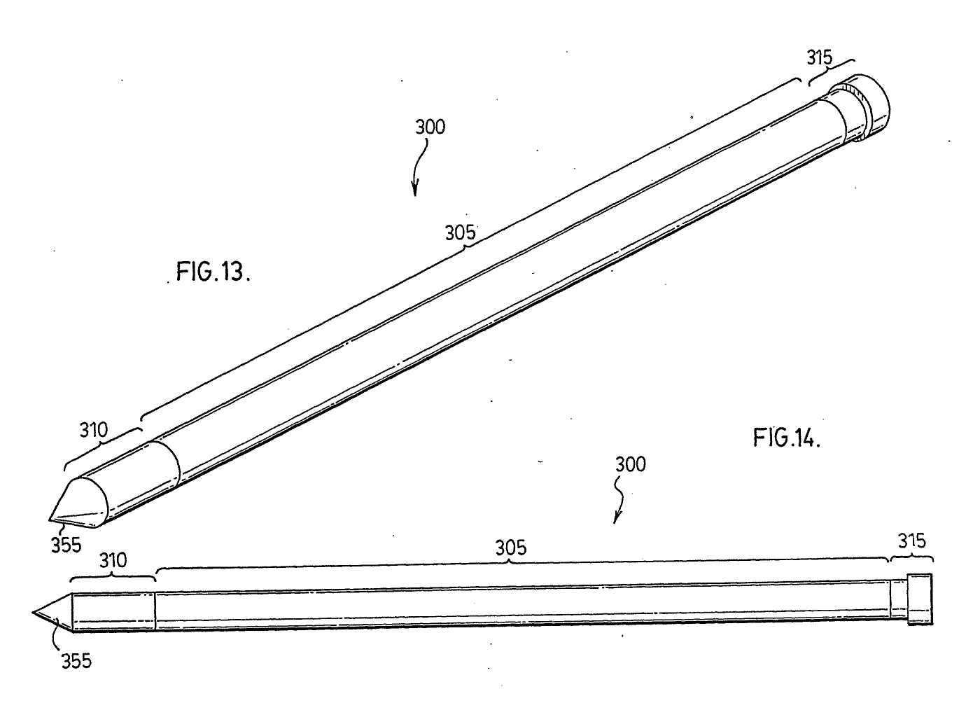

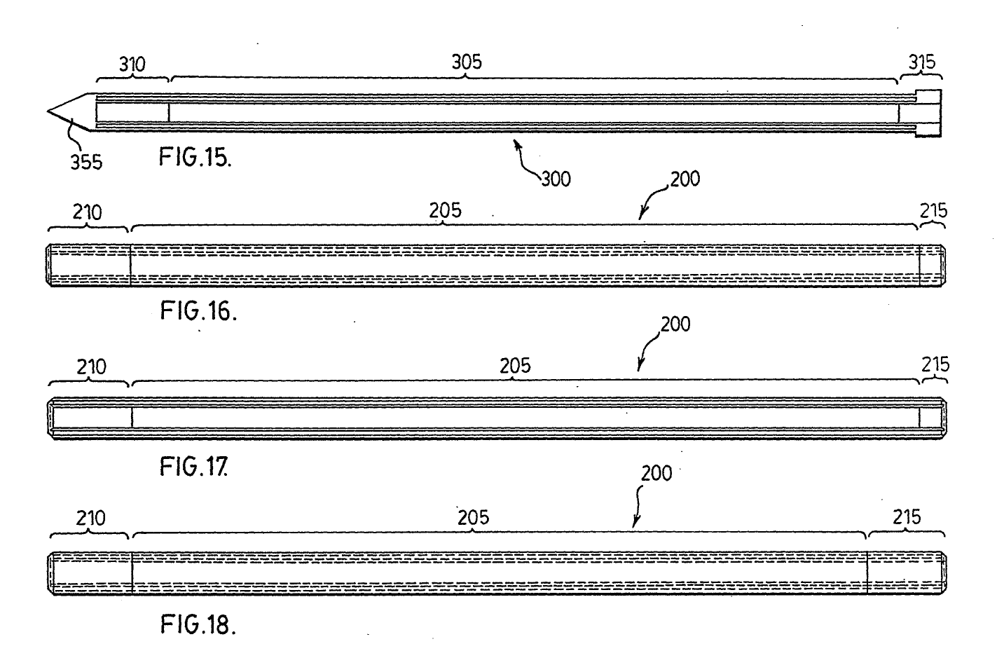

With reference to FIGS. 13-15, there is illustrated excimer radiation lamp assembly 300.

The principal modification in excimer radiation lamp 300 is the provision of a cone-shaped element 355 at the distal end of radiation opaque region 310. The provision of cone-shaped portion 355 facilitates self-location of radiation lamp assembly 300 during insertion thereof in a fluid treatment system.

Cone-shaped portion 355 may be made of quartz or any other suitable material that is durable in the environment in which radiation lamp assembly 300 is used.

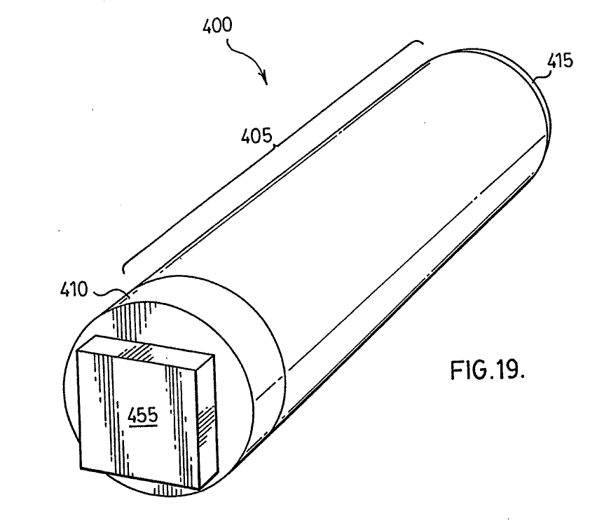

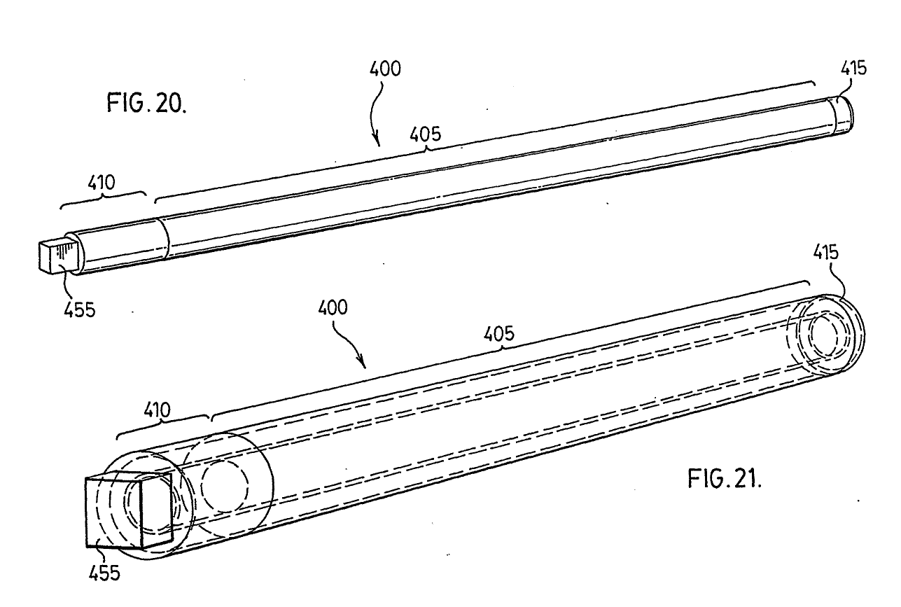

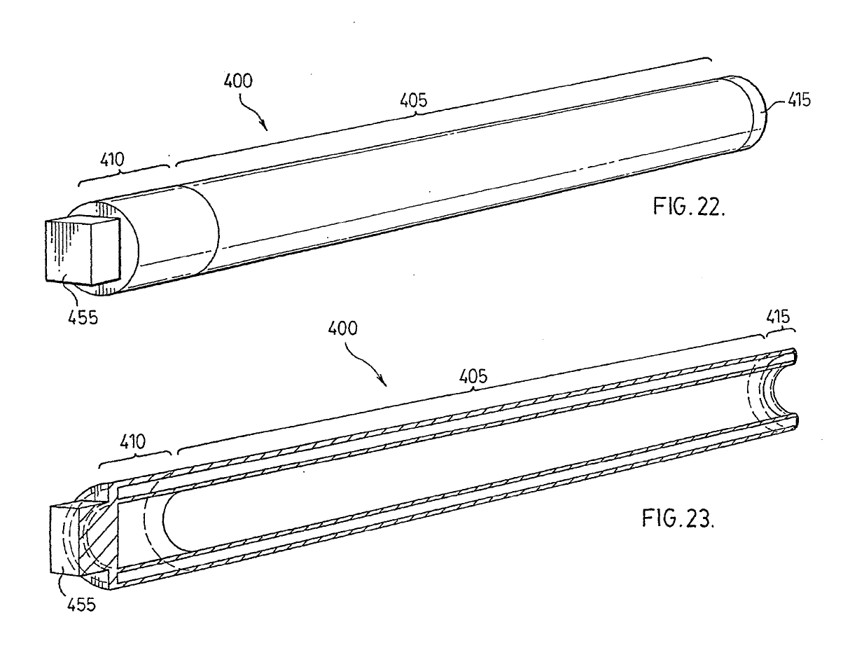

With reference to FIGS. 19-23, there is illustrated a radiation lamp assembly 400.

The principal modification in excimer radiation lamp 400 is the provision of a square shaped portion 455 at the distal end of radiation opaque region 410. The provision of square-shaped portion 455 facilitates self-location of radiation lamp assembly 400 during insertion thereof in a fluid treatment system.

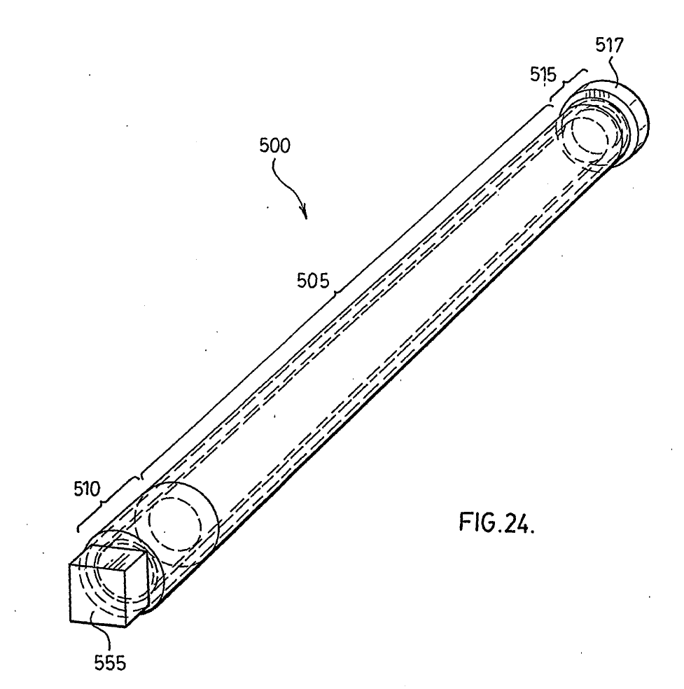

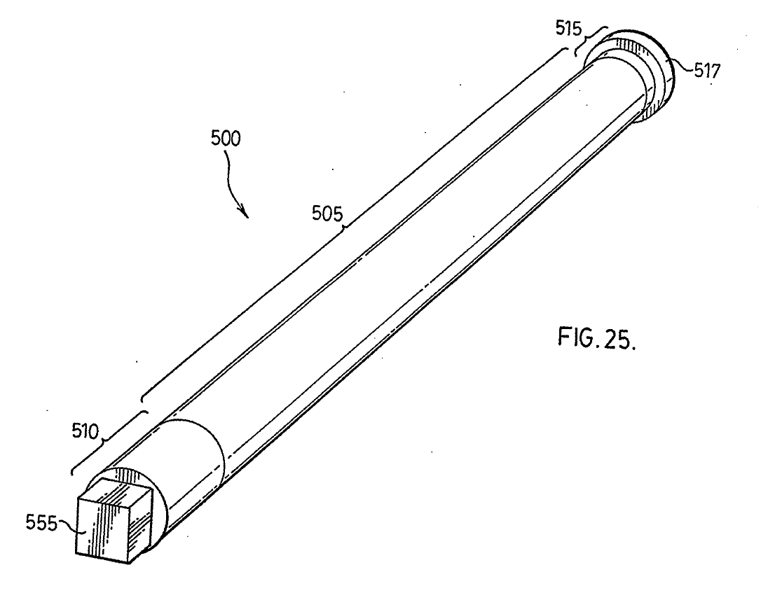

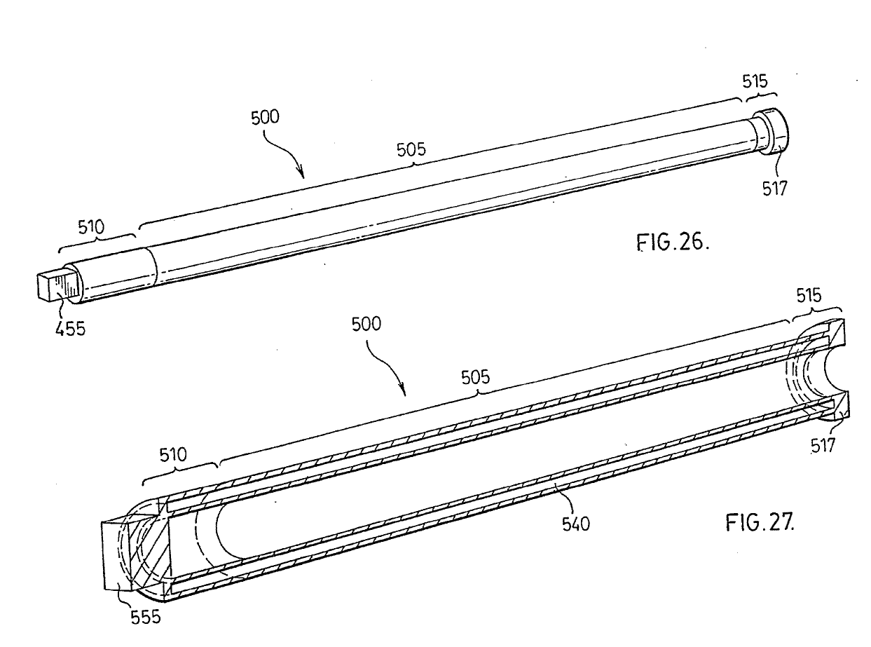

With reference to FIGS. 24-27, there is illustrated a excimer radiation lamp assembly 500. Excimer radiation lamp assembly 500 is similar to excimer radiation lamp 400 illustrated in FIGS. 19-23. The principal difference is annular element 517 has been added to lamp assembly 500, effectively to provide a double-keying capability to the lamp assembly. This ensures that the lamp be installed in a single manner only.

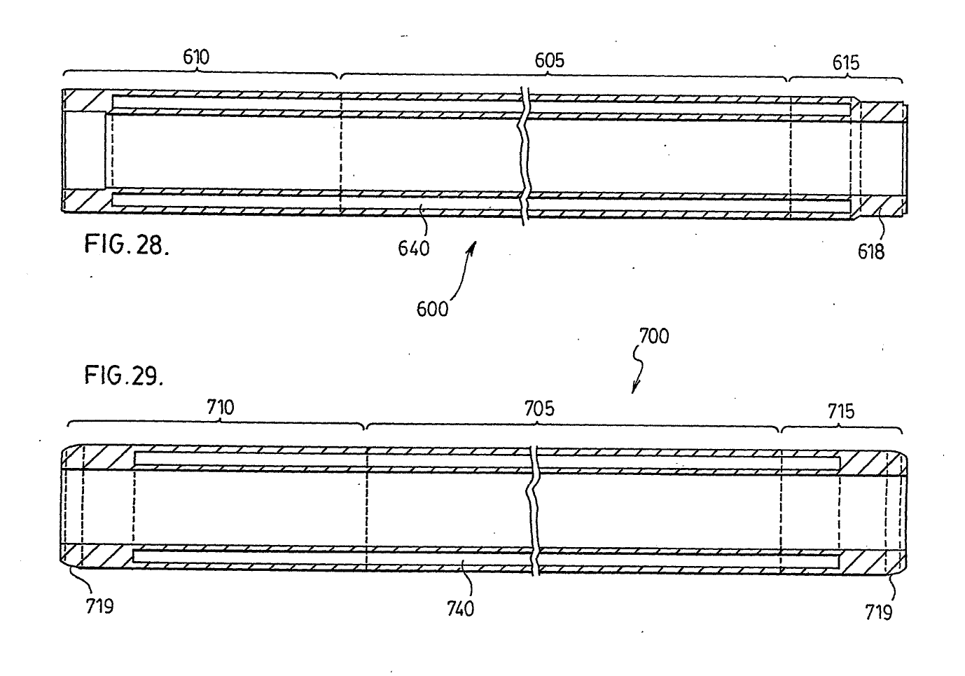

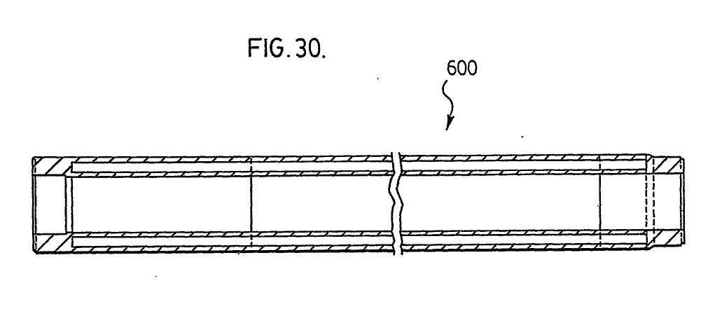

With reference to FIGS. 28 and 30, there is illustrated an excimer radiation lamp assembly 600.

The principal modification from the prior embodiments to excimer radiation lamp assembly 600 is the provision of a step-down portion 618 which serves to provide a “keying” function as described above. In other words, rather than having an enlarged diameter at this portion of the radiation lamp assembly, a step-down portion is provided to achieve a similar goal.

With reference to FIG. 29, there is illustrated a excimer radiation lamp assembly 700.

As show, excimer radiation lamp assembly 700 includes a chamfered portion 719 at the end of each of radiation opaque regions 710 and 715. The provision of chamfered portion 719 facilitates combination of excimer radiation lamp assembly 700 to provide a substantially fluid tight seal when radiation lamp assembly 700 is used in a fluid treatment system.

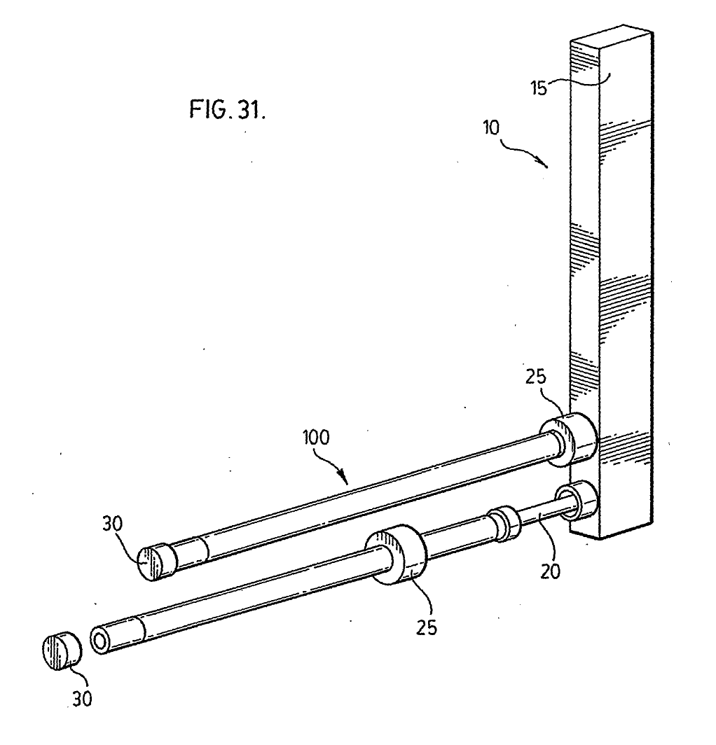

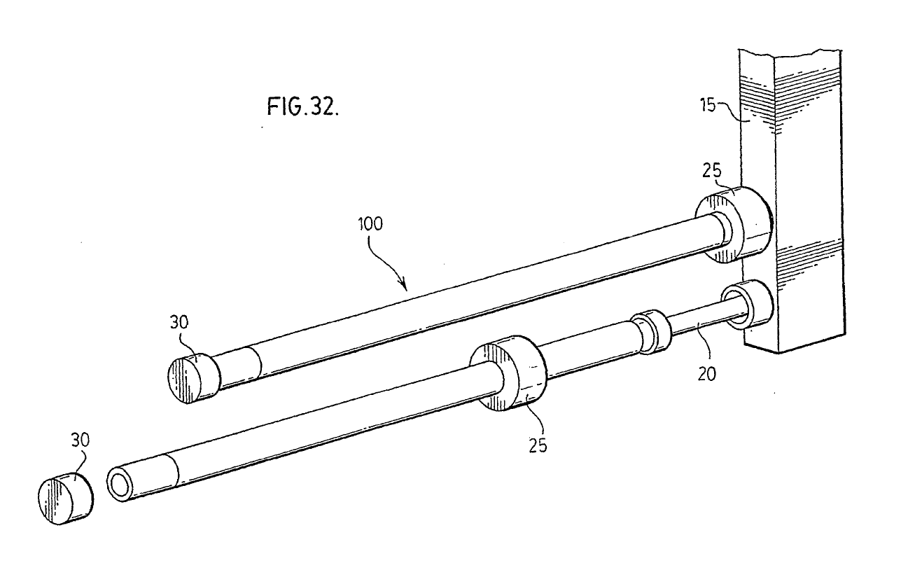

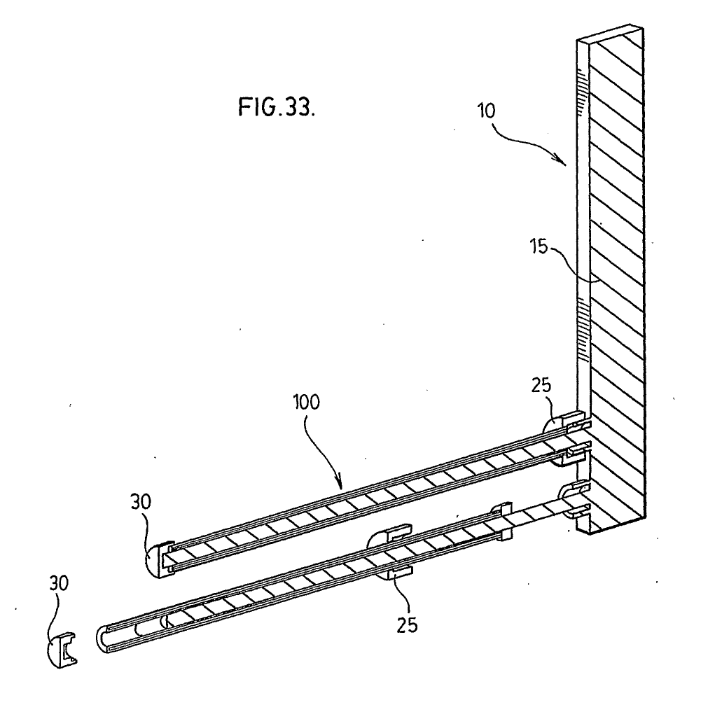

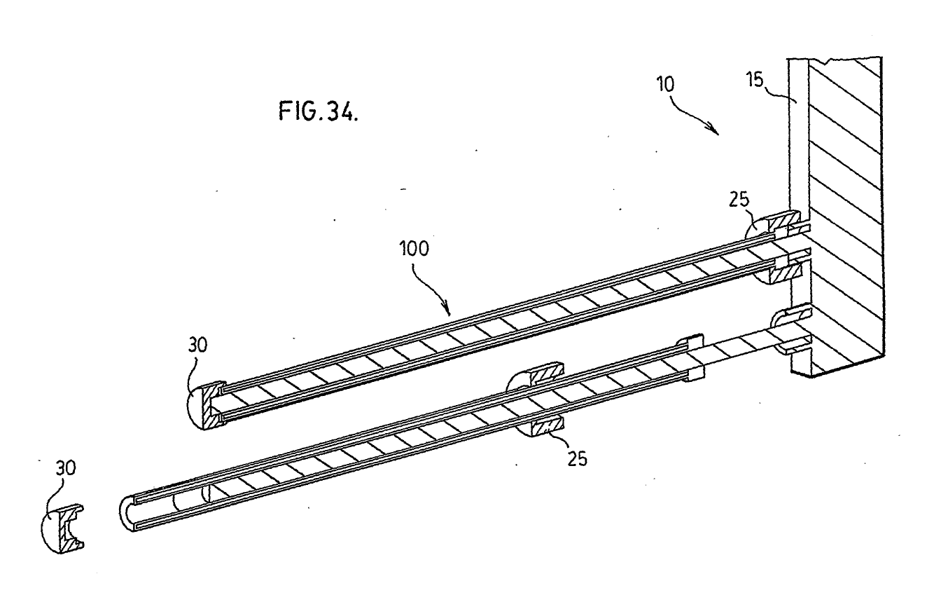

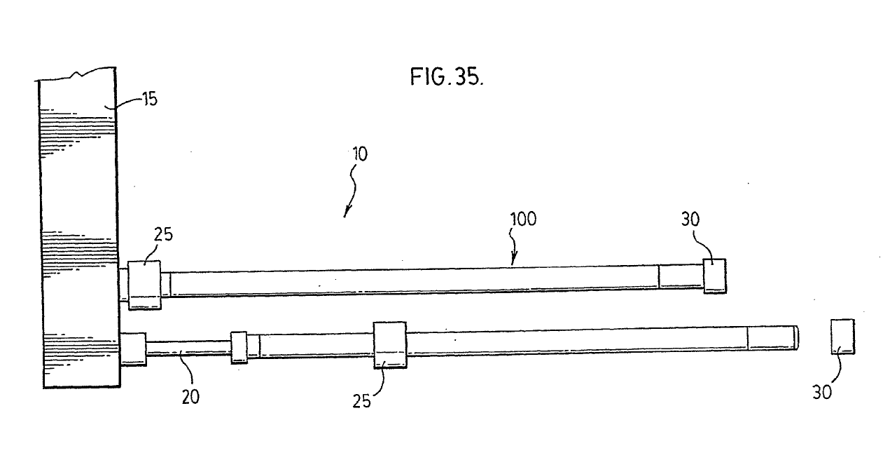

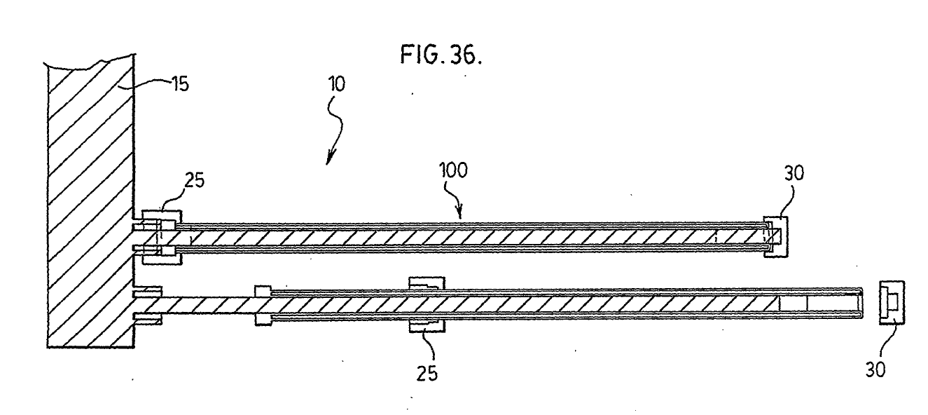

With reference to FIGS. 31-36, there is illustrated various embodiments of radiation source modules incorporating any of excimer radiation lamp assemblies 100,200,300,400,500,600,700.

Thus, there is shown a radiation source module 10 which is generally similar in design to the module shown in the U.S. Pat. No. 5,418,370—i.e., the radiation source is generally cantilevered with respect to a single support element 15.

When implementing a excimer radiation lamp assembly in a fluid treatment radiation source module such as module 10, a center electrode 20 is affixed to support element 15. Thereafter, the excimer radiation source assembly (excimer radiation source assembly 100 is shown as an example) is disposed over center electrode 20 and affixed thereto via a coupling nut 25 and a cap element 30. While FIGS. 31-36 do not show the detail of O-rings and other sealing elements, the selection and use of O-rings and other sealing elements is within the purview of a person of skill in the art.

All publications, patents and patent applications referred to herein are incorporated by reference in their entirety to the same extent as if each individual publication, patent or patent application was specifically and individually indicated to be incorporated by reference in its entirety. For greater certainty, two copending U.S. provisional patent applications 60/752,026 (Gowlings Ref: T8469434US) and 60/752,025 (T8469435US), both filed on Dec. 21, 2005 in the names of the present inventors, are each incorporated herein by reference.

1-7. (canceled)

8. A liquid immersible elongate excimer radiation lamp assembly having a longitudinal dimension, the assembly comprising:
a first end and a second end opposed to the first end;
a first region interposed between the first end and the second end for emission of a radiation having a prescribed wavelength; and
a second region juxtaposed with respect to the first region, the second region being radiation opaque or for emission of radiation different than the prescribed wavelength;
wherein the first end has at least one cross-sectional dimension different than that of the second end.

9. The lamp assembly defined in claim 8, wherein the first region is disposed asymmetrically with respect to a mid-point of the longitudinal dimension.

10. The lamp assembly defined in claim 8, wherein the first region is disposed symmetrically with respect to a mid-point of the longitudinal dimension.

11. The lamp assembly defined in claim 8, wherein the at least one cross-sectional dimension comprises a diameter of the first end or the second end.

12. The lamp assembly in claim 8, wherein the second region comprises a radiation opaque coating material applied to the lamp assembly.

13. The lamp assembly in claim 8, wherein the first region comprises a pair of ends constructed of a radiation transparent material.

14. A radiation source module comprising a first support element and at least one excimer radiation lamp assembly as defined in claims 8 connected at a first end thereof to the first support element.

15. (canceled)
